(19) United States
(12) Patent Application Publication
(10) Pub. No.: US 2011/0110985 A1
(43) Pub. Date: May 12, 2011

(54) FUNCTIONALIZED NANOMATERIALS FOR CHELATION THERAPIES AND SORBENT DIALYSIS OF TOXINS

(75) Inventors: Glen E. Fryxell, Kennewick, WA (US); Charles Timchalk, Kennewick, WA (US); Raymond S. Addleman, Benton City, WA (US); Wassana Yantasee, Richland, WA (US)

(73) Assignee: BATTELLE MEMORIAL INSTITUTE, Richland, WA (US)

(21) Appl. No.: 12/613,998

(22) Filed: Nov. 6, 2009

(51) Int. Cl. A61K 9/00; A61K 33/00; A61P 39/04

(52) U.S. Cl. 424/400; 424/600

(57) ABSTRACT

A therapy agent is disclosed that is made up of a functionalized nanomaterial that provides solutions to current problems facing the field of chelation therapies and dialysis of metals, radionuclides, and metabolic wastes. Through the coupling of groups tailored to selectively capture specific toxins and rigid porous backbone structures (e.g., mesoporous silica and mesoporous carbon), suitable materials that are highly effective and fast at capturing toxins (metals, radionuclides, and metabolic wastes) in the presence of competing ions and proteins. These materials may be embodied in a variety of treatment devices which allow for treatment and removal of these target materials through a variety of methodologies including oral, dermal and dialysis pathways.

### STATEMENT REGARDING RIGHTS TO INVENTION MADE UNDER FEDERALLY-SPONSORED RESEARCH AND DEVELOPMENT

This invention was made with Government support under Contract DE-AC05-76RLO1830 awarded by the U.S. Department of Energy. The Government has certain rights in the invention.

### BACKGROUND OF THE INVENTION

Exposure to toxic metals like cadmium (Cd), lead (Pb), mercury (Hg), and arsenic (As) is known to induce various diseases that are detrimental to human health. These heavy metals have a high affinity for thiol (—SH) groups, which can inactivate many enzymatic reactions, amino acid, and sulfur-containing antioxidants. Heavy metals are also believed to be responsible for the formation of free radicals and increased oxidative stress, which may be linked to various chronic diseases. For instance, Hg, even at low concentrations, is believed to be an environmental risk factor for cardiovascular disease. And, heavy metals may displace zinc, copper, and other essential metals and interfere with metalloenzyme functions, bone growth, and healing. Once introduced into the environment, heavy metals are not broken down, but can persist for a long time in air, water, and soil, thereby becoming sources for continued environmental exposure. Heavy metals can cause irreversible toxicity if not treated properly and in a timely manner.

Presently ethylenediamine-tetraacetate (EDTA) and meso-2,3-dimercaptosuccinic acid (DMSA) are FDA-approved liquid chelating agents for treatment of heavy metal poisoning. These agents bind metals in the blood and facilitate urinary and fecal excretion of the metals. EDTA is approved for the treatment of toxic metal (e.g., Pb) poisoning in adults, while DMSA is approved for the treatment of Pb poisoning in children whose blood Pb levels are >45 μg/dL. Sodium 2,3-dimercaptopropane-1-sulfonate (DMPS) given orally or intravenously has been used widely in Europe for chelation therapy of heavy metals (primarily Hg), although this therapy has not been approved by the FDA for use in the United States. However, these chelating agents still have important limitations. The intravenous EDTA chelation therapy requires multiple treatments (e.g., 3-4 hours, one to three times a week, for about 30 treatments at specialty clinics) and is costly. The DMSA is administered orally, thus is more convenient, safer, and less invasive but considered less effective (e.g., yielding less cumulative Pb excretion) than the intravenous EDTA. The side effects of the current chelating agents include: depletion of the body essential minerals (e.g., Zn, Cu, Fe, and Ca), redistribution of the metals to the brain, disturbing the gastrointestinal function, and skin rashes. Safer alternative oral delivered chelating agents for toxic metals have been recently available such as modified pectin from citrus fruits, alginate, and liquid zeolite. However, these materials lack a high affinity and specificity for heavy metals, and are prone to fouling and deactivation. Therefore, better chelating agents are needed for faster, safer, and more efficient removal of the toxic metals.

Likewise, chelation therapies for radionuclides using diethylenetriaminepentaacetic acid (DTPA) as a chelating agent have typically been limited for the following reasons: (1) DTPA is not specific for radionuclides over other essential minerals (e.g., Zn, Mg, Mn), which can lead to potential adverse side effects; (2) DTPA is not highly effective at the recommended daily dose. Therefore, it must be administered daily for an extended period (e.g., for years); (3) although Ca-DTPA is 10-fold more effective than Zn-DTPA when given in the first 24 hours, it is contraindicated for persons who have kidney diseases or bone marrow depression, are pregnant, or younger than 18; (4) DTPA is not approved for use with uranium (U); and (5) DTPA is not recommended for chelation of neptunium (Np) since it forms an unstable complex, which may increase Np deposition in bone. Insoluble Prussian Blue (ferric hexacyanoferrate) is also given orally to decorporate radiocesium (Cs) and radiothallium (Tl), but is known to bind to essential electrolytes like sodium (Na) and potassium (K). And, presently there are no effective chelating agents for radioactive cobalt (Co). Thus there is a need for better chelating agents than those currently approved by the FDA in terms of: 1) lower toxicity, 2) higher binding affinity and binding selectivity for target toxins over non-target species, 3) greater sorption capacity, 4) rapid sorption rate, 5) a favorable benefit-to-risk ratio, and 6) less cost. The present invention meets these needs.

Additional advantages and novel features of the present invention will be set forth as follows and will be readily apparent from the descriptions and demonstrations set forth herein. Accordingly, the following descriptions of the present invention should be seen as illustrative of the invention and not as limiting in any way.

### SUMMARY OF THE INVENTION

The present invention is a functionalized nanomaterial that provides solutions to current problems facing the field of chelation therapies and dialysis of metals, radionuclides, and metabolic wastes. By coupling groups that are tailored to selectively capture specific toxins and rigid porous backbone structures (e.g., mesoporous silica and mesoporous carbon), suitable materials have been developed that are highly effective and fast at capturing toxins (metals, radionuclides, and metabolic wastes) in the presence of competing ions and proteins. These materials may be embodied in a variety of treatment devices which allow for treatment and removal of these target materials through a variety of methodologies including oral, dermal and dialysis pathways. State-of-the-art sorbent dialysis and hemoperfusion methods still rely on activated carbon and zirconium phosphate for removal of metal cations. Functionalized nanoporous materials of the invention described herein are variably configurable into a 3-dimensional architecture having specific pore sizes and interfacial chemistries that allow for specific attachment, and thus provide advantages that traditional sorbents like activated carbon cannot. Various embodiments of these materials have shown superior sorption properties compared to conventional materials.

In one embodiment of the present invention, a therapeutic sorbent for removing materials from a biological system includes a material having a rigid, porous backbone coupled to at least one ligand configured to attach to a particular preselected target. Preferably, the rigid porous backbone is a mesoporous material such as a mesoporous silica, mesoporous carbon, or chemically modified activated carbon. These backbones are coupled to a ligand or functional group that is specific for binding to a particular target material. Preferably, these ligands are organic groups, however, there is no specific requirement that they be such. For example, in one embodiment, an inorganic phosphate ligand can be coupled to mesoporous TiO₂ to bind actinides. Thus, no limitations are intended. In other embodiments, various ligands configured to attach to various targets may be alternatively coupled to the same backbone. Depending upon the needs of the user, a multiplicity of such ligands may be utilized, with each ligand or groups of ligands individually configured to attach to a different target material. Examples of particular targets include, but are not limited to, heavy metals, radionuclides, and biological wastes among others. In particular, Cd, Pb, Hg, Tl, As, Gd, phosphate, U, Pu, Am, Cs, and Co have been utilized as targets in various embodiments; however other embodiments for other target materials are also contemplated within the scope of the present invention.

In another embodiment of the present invention an insoluble therapeutic chelating agent for removing heavy metals and radionuclide materials from a biological system includes a material having a rigid porous back bone coupled to at least one ligand configured to attach to a particular preselected target such as those described above. This insoluble therapeutic chelating agent maybe embodied in an oral delivery device, or may be otherwise alternatively configured so as to allow for a desired method of administration. Various inventive methods demonstrating the implementation of the present methods are shown and described hereafter.

Oral Chelation Therapy. In one embodiment, materials of the present invention are used as oral drugs to minimize absorption of ingested harmful chemicals through the gut to the human body and to reduce the body level of the toxins that undergo enterohepatic recirculation. These sorbents are not absorbed across the gut into the bloodstream, and thus are considered safer than chelating agents that do. Thus, these sorbents are capable of capturing toxins in the bloodstream that migrate across the gut membrane intro the gastrointestinal fluids. Each sorbent is designed to capture specific metals. Thus, there is less chance that non-target essential metals will be captured. They are also easy to administer and are safe enough for use in an ongoing basis, which will prevent the bounce back of serum metal levels and enables their use for prophylactic purposes. (e.g., to maintain low body levels of mercury for those who have fish and seafood as regular diet). Oral chelation therapy can also be effective for removal of inhaled substances because of the body's natural processes of expelling materials from the lungs into the digestive system as well as the two way transport of materials through the walls of the gut.

Sorbent Hemoperfusion. When used in hemoperfusion devices, functionalized nanomaterials can remove toxins in blood that have been absorbed systemically from all routes of exposure (oral, dermal and inhalation), which decreases the burden on the kidneys for clearing the toxic metal-bound liquid chelating agents. Some metals are not dialyzable since they bind to the protein components of blood, making removal with hemoperfusion using sorbents more effective than dialysis. The functionalized nanomaterials are designed to capture specific metals, thus less chance to capture non-target essential metals in blood. Also hemoperfusion using functionalized nanomaterials allows rapid removal of toxins before they leave intravascular space to other target organs (e.g., once dissociated Gd from Gd contrast agents leave intravascular tree, they deposit in skin tissues, a mechanism believed to trigger NSF disease).

Sorbent Dialysis. Functionalized nanomaterials can rapidly and selectively capture metals from dialysate and can be regenerated, thus they enable the development of personal sorbent dialysis devices based on sorbent dialysis technology. Sorbent dialysis exploits the sorbent cartridge that makes the system both simple and regenerative, unlike conventional hemodialysis. Spent dialysate from a dialyzer flows through the sorbent cartridge where the waste is removed and the regenerated dialysate is recirculated, thus minimizing the volume of dialysate needed. Thus, the sorbent dialysis system is simpler, generates less waste, and is more portable than conventional hemodialysis. Sorbent dialysis consumes less power because there is no need to purify and sterilize tap water used to make large quantities of the dialysate. There is also no need for dialysis machines to pump and heat large volumes of dialysate. Thus, sorbent dialysis is a necessary step toward next-generation personal dialysis devices, which are compact and portable.

In addition to these embodiments and applications, these materials may also be included in dermal applications, as well as to other applications. The purpose of the foregoing abstract is to enable the United States Patent and Trademark Office and the public generally, especially the scientists, engineers, and practitioners in the art who are not familiar with patent or legal terms or phraseology, to determine quickly from a cursory inspection the nature and essence of the technical disclosure of the application. The abstract is neither intended to define the invention of the application, which is measured by the claims, nor is it intended to be limiting as to the scope of the invention in any way.

Various advantages and novel features of the present invention are described herein and will become further readily apparent to those of ordinary skill in the art from the following detailed description. In the preceding and following descriptions, preferred embodiments of the invention are shown that illustrate the best mode contemplated for carrying out the invention. As will be realized, the invention is capable of modification in various respects without departing from the invention. Accordingly, the drawings and description of the preferred embodiment set forth hereafter are to be regarded as illustrative in nature, and not as restrictive.

### BRIEF DESCRIPTION OF THE DRAWINGS

FIG. 1a shows a solid chelating sorbent that comprises an ordered mesoporous silica backbone with ligands for binding specific target species.

FIG. 1b shows one embodiment of the present invention.

FIG. 1c shows a second embodiment of the present invention.

FIG. 1d shows a third embodiment of the present invention.

FIG. 1e shows a fourth embodiment of the present invention.

FIG. 2 shows the affinity of various target materials for various sorbents include embodiments of the present invention.

FIG. 3a shows the effect of ionic strength on affinity of various target metals to one embodiment of the present invention.

FIG. 3b shows the affinity of various target metals in various synthetic intestinal fluids to one embodiment of the present invention.

FIG. 4 shows the kinetics of Hg and Cd in synthetic gastric fluids when being treated by one embodiment of the present invention.

FIG. 5 shows the adsorption isotherm of Hg in synthetic gastric fluid to one embodiment of the present invention.

FIG. 6 shows the adsorption isotherm of Cd in synthetic intestinal fluid to one embodiment of the present invention.

FIG. 7 shows the adsorption isotherm of As(II) in synthetic intestinal fluid to one embodiment of the present invention.

FIG. 8 shows the results of an uptake study of one embodiment of the present invention in a simulated cell environment.

FIG. 9 shows a fifth embodiment of the present invention.

FIG. 10 shows the adsorption capacity of one embodiment of the invention at a preselected pH.

FIG. 11 shows the adsorption capacity of one embodiment of the present invention at a second preselected pH.

FIGS. 12a-12c show the amount of a target material in various tissues under various testing protocols.

FIGS. 13a-13c show the amount of a target material in various urine samples under various testing protocols.

FIGS. 14a-14c show the amount of a various target materials in fecal samples collected under various testing protocols.

FIG. 15 shows the estimated AUC (radioactivity) curve in rats tested in various testing protocols.

### DETAILED DESCRIPTION

The following description includes the preferred best mode of one embodiment of the present invention. It will be clear from this description of the invention that the invention is not limited to these illustrated embodiments but that the invention also includes a variety of modifications and embodiments thereto. Therefore the present description should be seen as illustrative and not limiting. While the invention is susceptible of various modifications and alternative constructions, it should be understood, that there is no intention to limit the invention to the specific form disclosed, but, on the contrary, the invention is to cover all modifications, alternative constructions, and equivalents falling within the spirit and scope of the invention as defined in the claims.

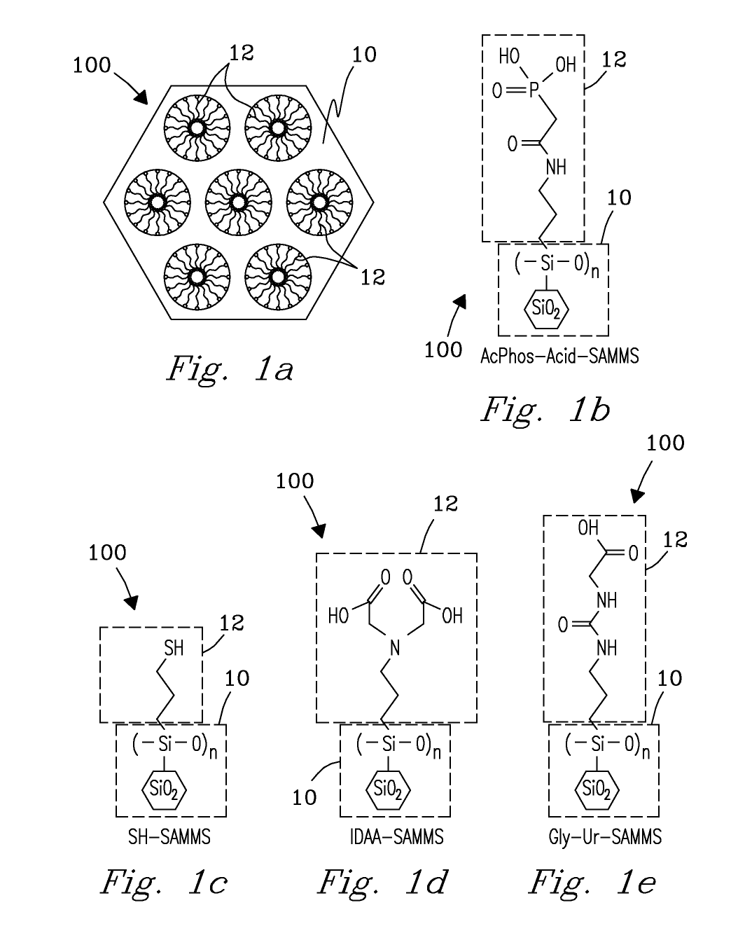

FIG. 1a shows one embodiment of the present invention. The present invention is a chelating sorbent 100 that includes a rigid, porous backbone material 10 that is functionalized with specific chemically-selective ligands 12 or binding sites 12. Ligands 12 of chelating sorbent 100 provide attachment to a specific target material. In the present embodiment, sorbent 100 is composed of ligands 12 that collectively form self-assembled monolayers on the mesoporous supports (SAMMS™, a registered trademark of Steward Advanced Materials, Inc., Chattanooga, Tenn., USA) material 10, but is not limited thereto. Mesoporous supports material 10 offers very large surface area (>200 m²/g) and functionality that has been fine-tuned to selectively capture heavy metals, including, e.g., actinides, lanthanides, iodine, cesium, and oxometallate anions. Sorbent 100 can be prepared in various forms for ingestion or delivery into the body, including, but not limited to, e.g., pills, tablets, capsules, shakes, powders, or other forms that allow the sorbent to delivered to the target area of the body. No limitations are intended.

FIGS. 1b-1e present various chemical structures of representative ligands 12 that form self-assembled monolayers on mesoporous silica (SAMMS) support materials 10 tested in this study. Chelation of As, Cd, Hg, and Pb from synthetic GI fluids was evaluated using these materials. In one embodiment of the present invention, chelating sorbents for capturing As, Cd, Hg, and Pb in gastrointestinal (GI) fluids are described. As will be described hereafter, these materials are better than existing materials, including, e.g., EDTA and DMSA, in terms of efficacy, convenient administration, and safe use. These sorbents can effectively capture toxic species from the GI system and can be used as oral drugs for: 1) limiting systemic absorption of ingested metals and 2) facilitating fecal excretion of ingested metals. These materials can also facilitate the elimination of heavy metals that have been previously absorbed into blood, excreted to the gut via bile, and reabsorbed again via enterohepatic circulation if not removed. In contrast to conventional liquid chelating agents, which are cleared by the kidneys as metal-chelate complexes, solid sorbents of the invention capture toxic metals which can then be cleared by fecal excretion, thus relieving the kidneys of a heavy metal burden that reduces the risk potential for renal failure.

### MATERIALS AND METHODS

SAMMS materials including, e.g., acetamide phosphonic acid (AcPhos)-SAMMS (FIG. 1b); thiol (SH)-SAMMS (FIG. 1c), iminodiacetic acid (IDAA)-SAMMS (FIG. 1d); and glycinyl-urea (Gly-Ur)-SAMMS (FIG. 1e), were tested. Synthesis and properties of SH-SAMMS is representative of this group of solid sorbents. SH-SAMMS is synthesized from a large pore mesoporous silica material 10, i.e. MCM-41, having a pore size of 80/55 Angstroms and a surface area of 1096 m²/g (as measured by Brunauer-Emmett-Teller (BET) nitrogen adsorption). Large pore MCM-41 is synthesized based on a protocol reported by Sayari et al. (“Applications of Pore-Expanded Mesoporous Silica. 1. Removal of Heavy Metal Cations and Organic Pollutants from Wastewater”, Chem. Mater. 2005, 17, 212-217). After thiol functionalization, the material has a BET surface area of 683 m²/g and a silane population of 2.1 silane/nm² (determined gravimetrically), or 2.3 silane/nm² (determined using thermogravimetric analysis).

Test matrices. Batch metal sorption experiments were performed with artificial gastric and intestinal fluids. The synthetic gastric fluid (SGF) and synthetic intestinal fluid (SIF) were prepared daily following the recommendations of the U.S. Pharmacopeia for drug dissolution studies in stomach and intestine, respectively. The SGF (pH 1.11) contained 0.03 M NaCl, 0.085 M HCl, and 0.32% (w/v) pepsin. The SIF contained 0.05 M KH₂PO₄; pH was adjusted to 6.8 with 0.2 M NaOH. Pancreatin was omitted from the SIF formula (unless specified otherwise). Modified Krebs-Henseleit buffer solution (pH 6.80) consisted of 118 mM NaCl, 4.7 mM KCl, 1.2 mM MgSO₄, 1.2 mM KH₂PO₄, 11 mM D-Glucose, 2.5 mM CaCl₂.2H₂O, and 25 mM NaHCO₃.

In vitro Caco-2 cell uptake. Caco-2 (i.e., human colon adenocarcinoma cell line) cells were seeded onto a semi-permeable membrane in a Transwell® polycarbonate membrane cell culture dish insert-receiver system (Corning Costar Corp., Cambridge, Mass., USA) for 21 days at 37° C. and 5% CO₂, and used to determine transport of metal-bound SH-SAMM across the human intestinal epithelium. The SH-SAMMS was pre-bound with 1.0 mg (Cd), 1.0 mg Hg, 1.0 mg (Pb), and 0.6 mg (As) per gram of SH-SAMMS prior to exposing the Caco-2 cells. The sorbent solid was suspended in a transport buffer (pH 7.4) consisting of 1.98 g/L of glucose, 10% (v/v) of 10× Hank's salt solution balanced with Ca and Mg, 0.01M of HEPES [4-(2-hydroxyethyl)-1-piperazineethanesulfonic acid] at the S/L ratio of 10 g/L. A 0.25 mL aliquot of this suspension was added to the apical (i.e., insert) side of the cell culture system, and 1.0 mL of the same buffer without metal-bound SH-SAMMS was added to the basolateral (i.e., receiver) side of the cell culture system. After 2 hr, the solution from the basolateral side was collected and diluted 10-fold in 2% of HNO₃ for ICP-MS analysis of the four metals (As, Cd, Hg, and Pb), as well as Si. The experiment was performed in triplicates with two controls (without metal-bound SH-SAMMS).

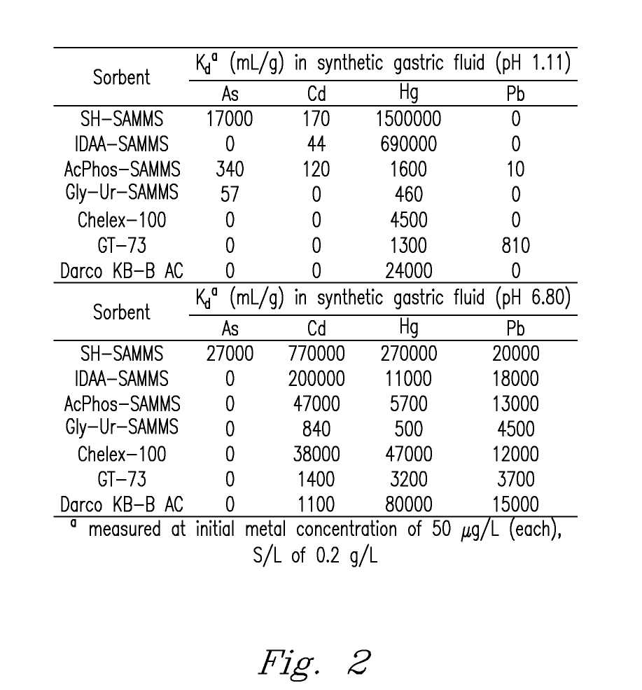

FIG. 2 is a table that summarizes the affinity of various sorbents for As, Cd, Hg and Pb ions measured in synthetic gastric fluid and synthetic intestinal fluids at a sorbent-to-liquid ratio (S/L) of 0.2 g/L. In the acidic synthetic gastric fluid (pH 1.11), most cation chelators could not capture the four metal species due to the protonation of the functional groups at low pH. The exception was SH-SAMMS, which could capture As and Hg, indicating the strength of the adduct between the soft thiol ligand and the soft metals As and Hg. IDAA-SAMMS and activated carbon (DARCO® KB—B Activated Carbon, Norit Americas, Inc., Marshall, Tex., USA) could capture Hg acceptably (K_{d}˜10⁴) but not as well as SH-SAMMS (K_{d}˜10⁵). At low pH, Cd and Pb (which are intermediate Lewis acids in terms of hard/soft acid-base theory) are not as effective as soft As and Hg at binding with the soft thiol ligand (K_{d}<10²).

In the synthetic intestinal fluid (0.05M H₂KPO₄, pH 6.8), SH-SAMMS is still the best for capturing all four metal ions with K_{d} of 10⁴ for As and Pb and 10⁵ for Cd and Hg. The IDAA ligand, which is a variant of EDTA, has been recognized as a powerful complexant. Having a relatively hard ligand, IDAA-SAMMS is better suited to capture intermediate Lewis acid transition metal cations like Cd and Pb much better than softer metals like As and Hg. The AcPhos-SAMMS, having phosphonic acid functionality, is generally better than Gly-Ur-SAMMS (having carboxylate functionality) at capturing metals in the high phosphate content system.

Although having the same functionality (e.g., thiol group), SH-SAMMS performed much better than the thiolated resin GT-73® (Rohm-Haas, Philadelphia, Pa., USA) for the metal capture in both synthetic fluids. This is because the SAMMS monolayer interface is highly ordered, making it possible for metal cations to interact with multiple thiol groups and therefore have a stronger binding interaction. Conversely, the polymer system of GT-73® is randomly ordered, and therefore the predominant interaction is with a single thiol group. IDAA-SAMMS generally performed better than the EDTA-based CHELEX-100® resin (Bio-Rad Laboratories, Inc., Hercules, Calif., USA) for the same reason. The SH-SAMMS performed better than other commercial resins. As expected, in the synthetic intestinal fluid, SH-SAMMS which binds with the metal ions via strong multidentate chelation reaction could capture the metal ions much better than the high surface area activated carbon (DARCO® KB—B activated carbon) having ligands (e.g., carboxylates, phenols, etc.) that undergo a less ordered, more random coordination with the metal ions just like the polymer-based ion exchange resins.

From the K_{d} values in FIG. 2, SH-SAMMS was identified as the best candidate for metal adsorption in the GI system and was subjected to further studies. The K_{d} values also suggest that, with SH-SAMMS, As and Hg can be removed from both stomach and intestinal fluids, while the majority of Cd and Pb will be removed in the intestine. To be effective as an oral treatment, the material must meet the following criteria; it must have high affinity for the target metals among the non-target metals in the relevant matrices, it must have sufficiently rapid metal binding rates, it must have large sorption capacity (e.g., not saturated with the non-target metals), it must not degrade in the GI tract and allow the release of the captured metal ions, it must continue to function in high concentrations of biomolecules and not be fouled by proteins, and it must not be damaged or be taken up by the cell lining of the intestinal tract. These criteria have been investigated using SH-SAMMS, as described hereafter.

Synthetic gastric and intestinal fluids used in this work were prepared according to formulas recommended by the U.S. Pharmacopeia for drug-dissolution studies in mammals (USP-XXVI, United States Pharmacopeial Convention Inc., Rockville, Md., USA, 26th Edition, 2003). However, in reality, the composition of GI fluid is highly dynamic and fluctuating, and is thus more complex than the simple phosphate buffer solutions recommended as a synthetic intestinal fluid. Bicarbonate buffer systems such as Hank's and Kreb's buffer solutions have been found to be better surrogates for intestinal fluids in some drug-dissolution studies. In addition, 0.2 M NaHCO₃ has been used as a synthetic intestinal fluid for in vitro toxic metal bioavailability studies.

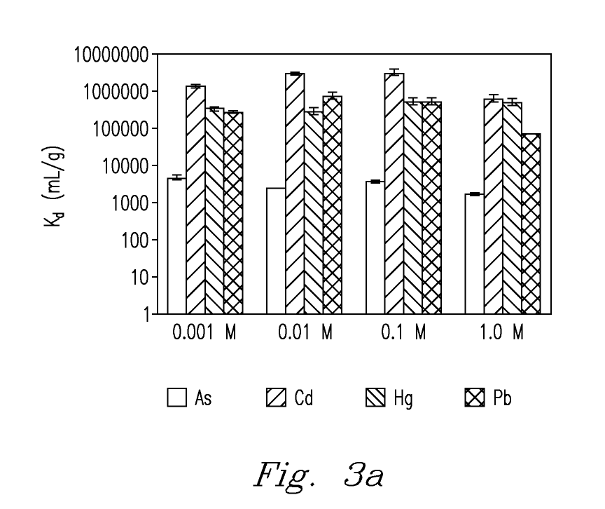

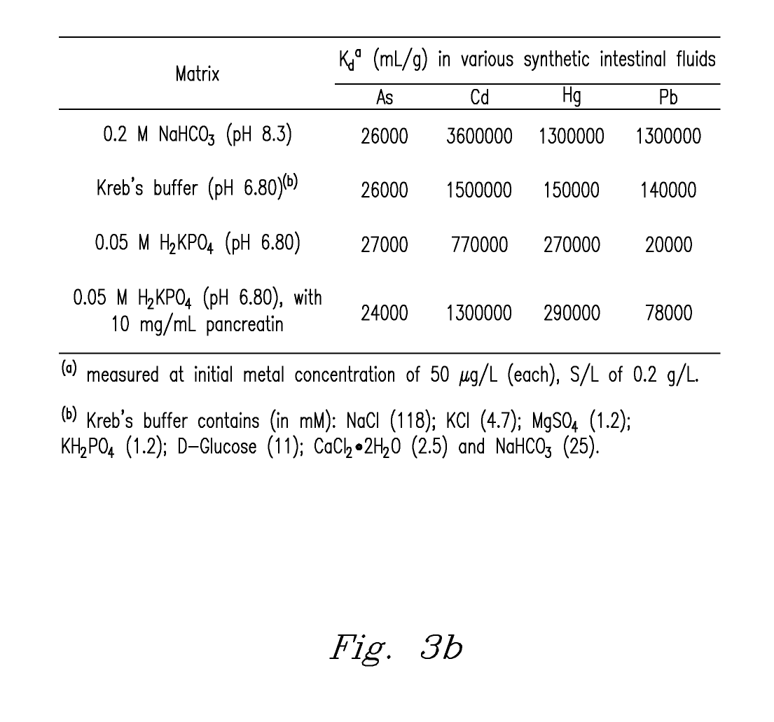

FIG. 3a shows the effect of ionic strength [i.e., by addition of sodium acetate (CH₃COONa) at pH 7.3] on the affinity (K_{d}) of adsorption by SH-SAMMS of various target metals, including, but not limited to, e.g., As, Cd, Hg, and Pb. Initial concentration of metal ions was 100 ug/L. Solid/Liquid (S/L) ratio was 0.2 g/L. FIG. 3b compares the affinity (K_{d}) of SH-SAMMS sorbent for these four metals measured in two synthetic intestinal fluid systems at a sorbent-to-liquid ratio (S/L) of 0.2 g/L: 1) a bicarbonate system, and 2) a phosphate system. Results suggest that SH-SAMMS can remove these four metals in both bicarbonate and phosphate systems equally well. Removal of Pb by SH-SAMMS is better in the bicarbonate system than the phosphate system, perhaps due to a weaker complex of Pb-carbonate than of Pb-phosphate. In the figure, the performance of SH-SAMMS materials within various pH ranges is also demonstrated. Various pH values were used in order to replicate pH conditions similar to what might be encountered within the various regions of the gastrointestinal tract (e.g., pH 1.0-3.0 in the stomach; pH 5.5-7.0 in the large intestine; pH 6.0-6.5 in the duodenum; and pH 7.0-8.0 in the jejunum and ileum). The affinity of Pb and Cd for SH-SAMMS follows a normal trend of cation metal binding on cation chelators, while the affinity for Hg was high across the whole pH range (K_{d}˜10⁶), revealing the robust nature of the SH-SAMMS adduct under both acidic and alkaline conditions.

Polymer resins such as those known in the prior art have been known to suffer from swelling and shrinking affected by variation in solution ionic strength, which may retard the therapeutic properties of the resin based drugs. Result shows that increasing the concentration of sodium acetate buffer from 0.001M to 0.1M did not significantly change the affinity of SH-SAMMS for the four metals. Consequently, variations in the ionic strength of the GI fluids are unlikely to significantly impact the chelation of these four metals by SH-SAMMS.

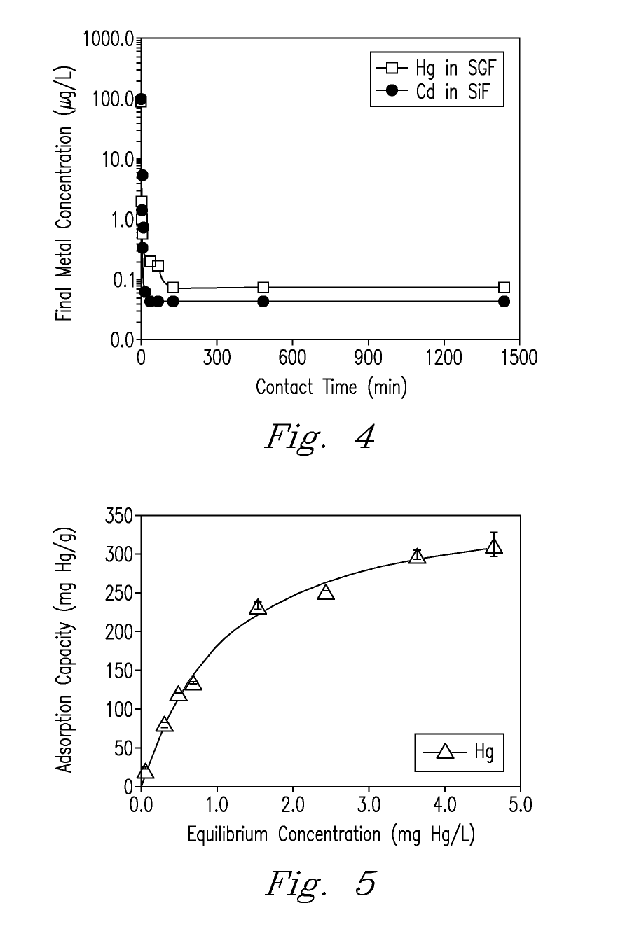

FIG. 4 shows the sorption kinetics of Hg in synthetic gastric fluid (SGF) and of Cd in synthetic intestinal fluid (SIF). Over 99% of Hg in SGF and Cd in SIF were removed after 3 minutes. This rapid sorption rate is owed to the rigid pore structure and mesopore size, which make all of the thiol binding sites available at all times, in contrast to swellable polymer ion exchange resins such as GT-73. From 2 to 24 hrs of contact time, the extent of sorption remains steady, indicating that there is no significant leaching of Hg and Cd back off of the laden sorbent, and no significant degradation of the materials in these two matrices (the behavior of these sorbents during the first 24 hours is of primary interest since when administered orally, they are excreted fecally after about a day).

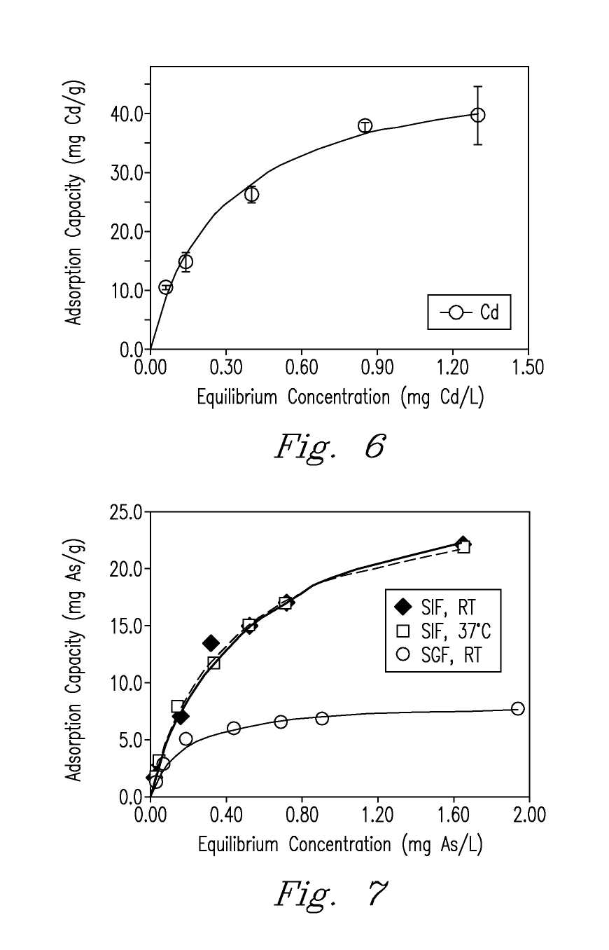

FIGS. 5-7 show adsorption isotherms of Hg in synthetic gastric fluid (SGF), Cd in synthetic intestinal fluid (SIF), and As in both SGF and SIF, respectively. The metals were tested in these matrices because the K_{d} (see FIG. 2) suggested that Cd would preferentially be removed in the intestine, while As and Hg would be removed in both the stomach and the intestine. All the data sets are represented well by a Langmuir adsorption model (R²>0.98) suggesting monolayer adsorption without precipitation of the metal ions out of the solutions at these conditions. The isotherm data indicates that in these synthetic matrices having high concentration of other ions, SH-SAMMS offers high uptake capacities owing to the selectivity of the material for the target metals.

In addition, these materials demonstrated good stability in these synthetic fluids. The wt % of Si dissolved per total mass of SH-SAMMS after 2 hrs of stirring in synthetic gastric fluid (pH 1.11) and synthetic intestinal fluid (pH 6.8) fluid were measured to be 0.2 and 2% (by weight), respectively. SH-SAMMS has a long shelf-life (some of the batches are over 5 years old but still maintain the metal binding performance), making it feasible for stockpiling with proper storage. In vivo testing of Caco-2 cells replicate many of the properties of the small intestinal epithelium and have been used in many studies to determine transport of chemicals across the human intestinal epithelium. In one set of experiments, Caco-2 cells cultured for 21-days in a transwell polycarbonate membrane culture dish were used to investigate the transport of SAMMS across the epithelial cells. After 30 min of suspension of SH-SAMMS (that was pre-bound with 1.0 mg (each) of Cd, Pb, and Hg and 0.6 mg of As per gram) in the transport buffer (pH 7.4), there was no detectable leaching of Cd, Pb, and Hg, and only small leachate of Si and As (0.1 wt % and 0.3 wt %, respectively). Thus most metals remained bound to the SAMMS material prior to adding it to the Caco-2 cells. The metal-bound SAMMS suspension was added to the Caco-2 transwell insert to obtain 0.0025 g of metal-bound SAMMS which corresponded to 1.4 mg of As and 2.5 mg (each) of Cd, Hg, and Pb.

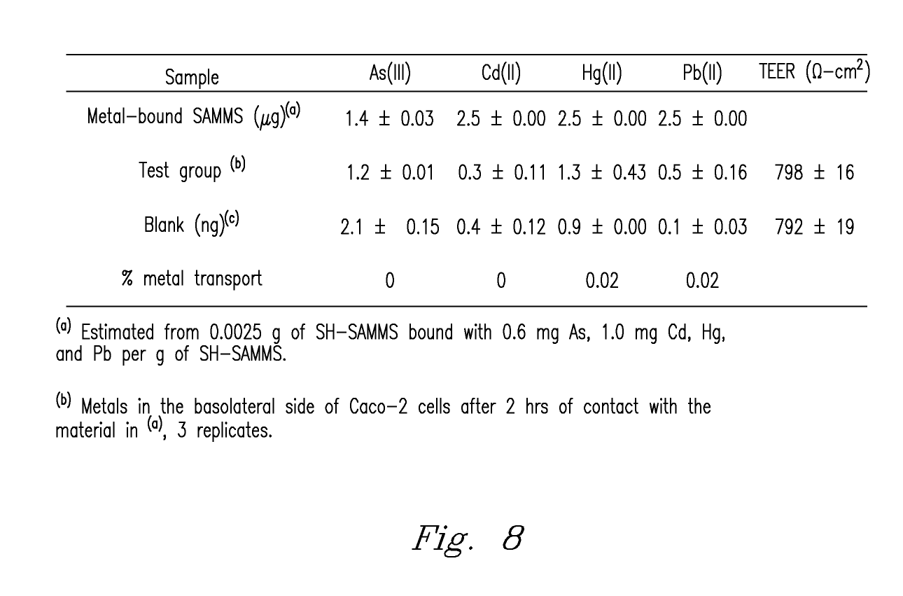

After 2 hours of incubation, there was no difference in concentrations of the four metals in the basolateral side between the test and the control groups (with no metal-bound SAMMS material added) and only low nanograms of metals were detected. TABLE 4 also shows that the percent (%) transport of metals across the Caco-2 monolayers per amounts added (from pre-binding with SH-SAMMS) was negligible, which indicated that once bound with SH-SAMMS, the metals were not released into the transport buffer. It also indicates that SAMMS was not taken-up by the Caco-2 cells, which is attributed to the relatively large particle size of SH-SAMMS (95% of the material is larger than 5 μm and the mean particle size is 22 μm). A series of DIC and fluorescence images, taken through the Z-axis of the cells after exposure to fluorescent dye-tagged SH-SAMMS for 3 hr, followed by fluorescence quenching by Trypan Blue reveal that large particles (>5 μm) remained on the cell surface, while smaller particles (1-2 μm) could enter the cell cytoplasm. No change in the morphology of the cells was detected in the presence of the larger particles, when compared with control cells (not shown). Thus for oral drug candidate, SH-SAMMS that is larger than 5 μm in size is recommended. It's worth nothing that although their particle size is large, SAMMS materials achieve high surface area through their high porosity. FIG. 8 also shows that there was no decrease in the resistance [i.e., as determined by Trans-Epithelial Electrical Resistance (TEER) measurements of cell viability (see, e.g., http://www.pharmaceutical-int.com/categories/teer-measurement/trans-epithelial-electric-resistance-teer-measurements.asp)] across the cell monolayers from those that were not exposed to metal-bound SH-SAMMS (792±19 Ω-cm²) as compared to those that were exposed (798±16 Ω-cm²). Thus, based on the resistivity, the SAMMS material did not disrupt the cell monolayer, and no cell damage was observed.

SH-SAMMS has proven to be effective in capturing organic metallic species such as methyl mercury (CH₃Hg⁺). The K_{d} values of SH-SAMMS for CH₃Hg⁺ in filtered river water at pH 2.0 and 8.1 were 170,000 and 88,000, respectively. Under the same testing conditions, K_{d} values for capture of inorganic Hg²⁺ were 640,000 and 190,000, respectively. River water is a preferred test matrix, as because CH₃Hg⁺ is formed in the environment via methylation process of inorganic Hg by microorganisms in sediments and is readily bioaccumulated in aquatic food chains. Once ingested, CH₃Hg⁺ is well absorbed (>90%) in humans. It is well distributed to all tissues in the body, and most importantly readily crosses the blood brain barrier where it can exert substantial neurotoxicity. Thus, it is of a substantially higher toxicity concern than its inorganic counterpart. Hence, SH-SAMMS that can effectively capture CH₃Hg⁺ will increase fecal excretion of Hg and minimize its bioaccumulation. Not only will CH₃Hg⁺ from ingested diets be eliminated, but the blood level of CH₃Hg⁺ would be reduced since it readily undergoes enterohepatic recirculation. In this regard, a SH-based resin has been shown to improve fecal excretion of CH₃Hg⁺ in rats and reduce blood level of CH₃Hg⁺ in the Iraq outbreak in early 1970s. The SH-SAMMS material would be much more effective than the resin based materials in term of binding affinity, capacity, and rate.

One of the drawbacks of EDTA chelation therapy is that it facilitates urinary excretion of essential minerals, especially Ca (by 2-fold on the day of chelation, compared to one day and two days prior to treatment) and Zn (by 18-fold). Hypocalcemia due to chelation therapy can eventually lead to cardiac arrest, and three deaths have been recently reported [http://www.cdc.gov/mmwr/preview/mmwrhtml/mm5508a3.htm].

Uptake of essential minerals by SH-SAMMS was tested in our laboratory using similar metal concentrations that may be encountered in human plasma, since metal concentrations in the gut were not available to us and can be largely dependent on diet. The synthetic gastric and intestinal fluids included the essential minerals Ca, Mg, Fe, and/or Mo at concentrations of 100 mg/L of Ca, 30 mg/L of Mg, 0.5 mg/L of Fe(III), and 0.5 mg/L of Mo. Intestinal fluid was simulated with Krebs buffer, which kept the metals soluble better than 0.05M H₂ KPO₄. Results showed SH-SAMMS did not remove significant quantities of these essential minerals. This observation follows Pearson's hard-soft acid-base theory (HSAB) that the soft thiol ligand will have very low affinity for hard metal cations like Ca, Mg, and Fe. When 0.5 mg/L of Zn and Cu was added to the synthetic intestinal fluid, the metals could be largely collected by 0.45 μm filters (even without SH-SAMMS), making it difficult to assess their uptake on SH-SAMMS. However, it is presumed that both Zn and Cu (which are intermediate Lewis acid metal cations according to the HSAB principle) will be captured by SH-SAMMS, but perhaps to a lesser extent than heavy metals. For example, SH-SAMMS has been shown to have a much lower affinity for Zn than for Hg in aqueous media. Liquid DMSA given orally to rats did not significantly change the concentrations of Ca and Zn in the carcass, nor those of Fe and Cu in the liver, kidney, or brain. Given that SH-SAMMS has a thiol functionality, it is expected to behave similarly to DMSA.

In vitro assessments suggest that SH-SAMMS has great potential as an oral drug for removing metals in the GI system. The chemical composition of SH-SAMMS suggests that it should be sufficiently safe to use in an ongoing basis, i.e., using repeated doses over an extended period of time to prevent “bounce-back” of serum metal levels. When metals are taken out of the blood, metals stored within soft tissues and hard tissues can re-equilibrate with the blood (slowly). So, if metals are removed from the blood with a single dose of sorbent, the blood concentration of metals can “bounce back” or re-equilibrate with concentrations of metals located within the soft tissues or hard tissues. In addition, SH-SAMMS can be used for preventive purposes, e.g., to maintain low body levels of mercury in persons who eat a regular diet, e.g., of fish and seafood. Metabolism, degree of gut absorption, biliary excretion, enterohepatic circulation, and native ligand binding of various metals will also affect the effectiveness of SH-SAMMS for the treatment of acute and chronic metal poisoning.

In other testing, examples of in vitro and in vivo testing (in a rodent model) demonstrate the efficacy and capacity of SAMMS to decorporate ¹³⁷Cs relative to Prussian Blue.

### Decorporation of Radionuclides

Cesium (Cs) and radiocesium (137Cs). For in vitro batch experiments, cesium (Cs⁺) was purchased as a standard solution at a concentration of 1000 mg/L in ˜2% HNO₃. For in vivo pharmacokinetic evaluation, two separate batches of cesium chloride (¹³⁷CsCl) were obtained from Amersham International (Amersham, UK) and ICN Isotope and Nuclear Division (Irvine, Calif.).

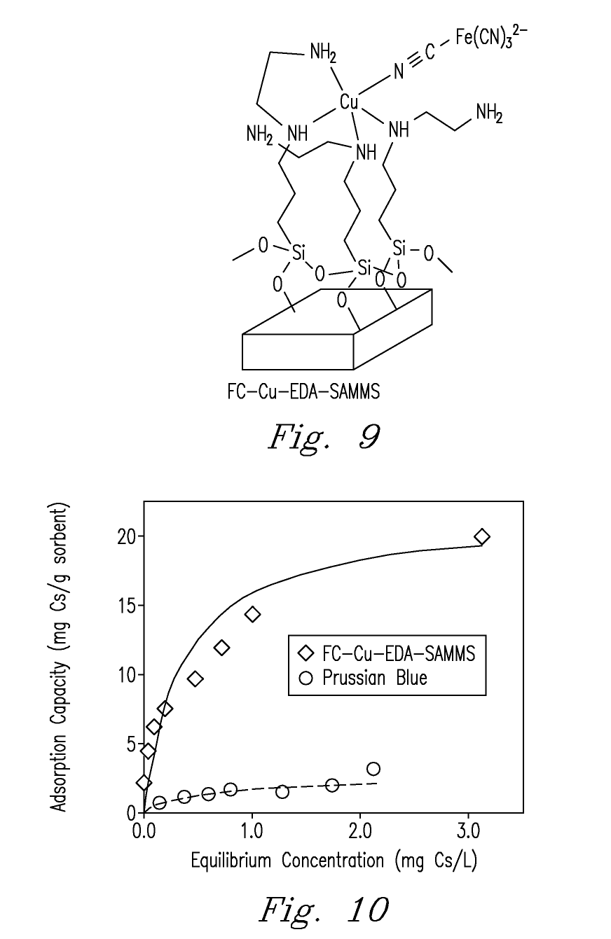

Sorbents. Synthesis of FC—Cu-EDA-SAMMS sorbent has been described by Lin et al. (in “Selective Sorption of Cesium Using Self-Assembled Monolayers on Mesoporous Supports (SAMMS)”, Environmental Science and Technology 2001, 35, 3962-3966), which reference is incorporated herein. The substrate was MCM-41 silica, with a surface area of 900 m²/g and a nominal pore size of 3.5 nm. Ethylenediamine (EDA)-terminated silane was deposited, in refluxing toluene, to produce EDA-SAMMS. Next, the EDA-SAMMS was treated with an excess of CuCl₂ in water, filtered and dried. The Cu-EDA-SAMMS was thermally cured in refluxing toluene (Dean-Stark trap) for 2 hours. The Carolina blue powder was collected by filtration and air-dried. Next, a solution of excess sodium ferrocyanide was prepared and the Cu-EDA-SAMMS was added with vigorous stirring. The suspension turned a deep violet color as the ferrocyanide anion reacted with the Cu-EDA complex. The FC—Cu-EDA-SAMMS was collected by filtration, washed with water and alcohol and air-dried. (Unless specifically noted the use of the acronym SAMMS will refer to FC—Cu-EDA-SAMMS). Insoluble Prussian Blue, Fe₄[Fe(CN)₆]₃, was purchased from Aldrich Co. FIG. 9 illustrates the chemical structure of FC—Cu-EDA-SAMMS.

Gamma Counting. Samples were each counted for 10 minutes using a shielded, well-type gamma counter (e.g., Wallac-1480 WIZARD® gamma counter, Perkin-Elmer, Waltham, Mass., USA). The counting efficiency for ¹³⁷Cs was 47% with minimal sample crosstalk (0.001%).

### In Vitro Experimental Design

K_{d} measurements. The metal sorption performance of SAMMS and Prussian Blue was evaluated in terms of the distribution coefficient (K_{d}, mL/g), which is a mass-weighted partition coefficient between the solid phase and liquid supernatant phase. Two test matrices were used: 1) a synthetic gastric fluid, which contained 0.03M NaCl, 0.085M HCl, and 0.32% (w/v) pepsin, prepared daily following U.S. Pharmacopeia recommendations for drug dissolution studies in stomach (USP, 1990); 2) a synthetic intestinal fluid, which contained 0.05M NaHCO₃, which has been used as an intestinal fluid simulant in other studies (see, e.g., Hamel et al., Sci. Total Environ. 243-244: 273-83; 1999; and Ellickson et al. Arch. Environ. Contam. Toxicol. 40: 128-35; 2001). The K_{d} values of Cs in synthetic gastric and intestinal fluid were measured in batch experiments with 50 ppb starting concentration of Cs and liquid per solid (L/S) ratio of 5,000 mL per gram of material. The suspension was shaken in a polypropylene bottle at a speed of 250 rpm for 2 hours at 37° C. After the batch contacts, metal-laden sorbents were filtered through 0.2 μm Nylon filters in a polypropylene housing. Both initial and final solutions (before and after the batch experiments) were analyzed by an inductively coupled plasma-mass spectrometer (Agilent ICPMS model 7500ce, Agilent Technologies, Inc., Santa Clara, Calif., USA). Measurements were carried out in triplicate and average values were reported.

Sorption isotherms. The sorption capacities of SAMMS and Prussian Blue for metal ions were measured in the same fashion as with the K_{d}, but the starting concentrations of Cs were varied in the solution until maximum sorption capacity was obtained. This was accomplished by using a large excess of metal ions to the number of binding sites on the sorbent materials (e.g., 0.1 to 5 mg/L of Cs at L/S of 10,000 mL/g).

### In Vivo Experimental Design

Treatment Group. Three experimental groups were evaluated. Group I (controls) received only ¹³⁷Cs by intravenous (iv) or oral administrations and were used to establish the oral bioavailability and clearance rate for ¹³⁷Cs. Group II established the stability of the ¹³⁷Cs-SAMMS adduct (pre-bound) and the rate of ¹³⁷Cs sequestration in vivo in the rat gut. Group III compared the initial efficacy of SAMMS vs. Prussian Blue to sequester ¹³⁷Cs following oral exposures.

Animals. For all studies, male Sprague-Dawley rats (291-341 g) with jugular vein cannulae were obtained from Charles River Laboratories, Inc. (Wilmington, Mass., USA). Rats were housed in plastic metabolism cages and were fed Purina Certified Rodent Chow® 5002 (Purina Mills, St. Louis, Mo., USA) ad libitum. Feed was withdrawn ˜6 hour prior to dosing and returned 3 hour post-dosing. Water was available ad libitum throughout the duration of the study. Blood was collected through the jugular vein cannula at 0.5, 1, 2, 3, 6, 12, 24, 48 and 72 hour post-dosing. Urine and feces were collected continuously, and sample collections were accumulated for 24, 48, and 72 hour post-dosing. All rats were euthanized at 72 hour postdosing and selected tissues were collected for analysis.

Dosing. The ¹³⁷Cs stock solutions were initially diluted to an acidic concentration of 0.01M HCl, then buffered with phosphate buffered saline (PBS) to make the dosing solutions. ICP-MS analysis of the dosing solutions indicated Cs concentrations of 58.3 μg/mL, 51.0 ng/mL and 53.2 ng/mL for Groups I, II and III, respectively. Radiological activity of these dosing solutions by gamma count was 8.14 kBq/mL, 18.5 kBq/mL and 17.8 kBq/mL, respectively. The average amount of ¹³⁷Cs and associated radioactivity administered to the rats for treatment Group I was 40.4 μg/kg and 5.5 kBq/kg, respectively. Whereas, for treatment Groups II and III, the average ¹³⁷Cs dose was ˜61 ng/kg, while the average amount of radioactivity administered were 22.6 kBq/kg and 20.4 kBq/kg, respectively. For Group II, the pre-bound ¹³⁷Cs-SAMMS was prepared by mixing the ¹³⁷Cs dose solution with an excess of SAMMS and allowing the solution to mix for 30 min at room temperature. The SAMMS was then filtered and the remaining supernantant was analyzed for radioactivity; which was at background levels (data not shown), indicating that all the ¹³⁷Cs was bound to the SAMMS. The pre-bound ¹³⁷Cs-SAMMS was then orally administered to rats as previously described. For Group III, 0.1 g of SAMMS or Prussian Blue was suspended in 1 mL of PBS which was then administered to rats by gavage.

Data Analysis. The time-course of ¹³⁷Cs was analyzed using non-compartmental methods. Peak concentrations of ¹³⁷Cs in blood (Cₘₐₓ) were determined by a visual analysis of the individual observed concentration-time data. The area under the blood concentration-time curve from 0-72 hour (AUC) was determined using Graphpad Prism®4 using the trapezoidal rule. Other than the calculation of mean standard deviation, no additional statistical evaluations were conducted.

### Results

### In Vitro

In this study, the sorption performance of complexed copper (II) ferrocyanide immobilized on mesoporous silica (FC—Cu-EDA-SAMMS) for Cs in a gastric and intestinal fluid simulant were evaluated in terms of adsorption affinity and capacity. The performance was also evaluated against insoluble Prussian Blue, which is considered the best commercially available sorbent for Cs, and also FDA-approved in 2003 for radioactive Cs and Tl decorporation therapies (FDA 2003).

The adsorption affinity of Cs on SAMMS and Prussian Blue has been investigated using synthetic gastric and intestinal fluid matrix simulants The sorption affinity is often represented in term of the distribution coefficient, K_{d} (in the unit of mL/g), The in vitro measured K_{d} for the SAMMS substantially exceeded the adsorption affinity of Prussian Blue in stimulants of gastric (˜29-fold) and intestine fluid (˜3-fold). These results indicate that the SAMMS material has excellent affinity for the Cs and exceeded the affinity of Prussian Blue under these in vitro experimental conditions.

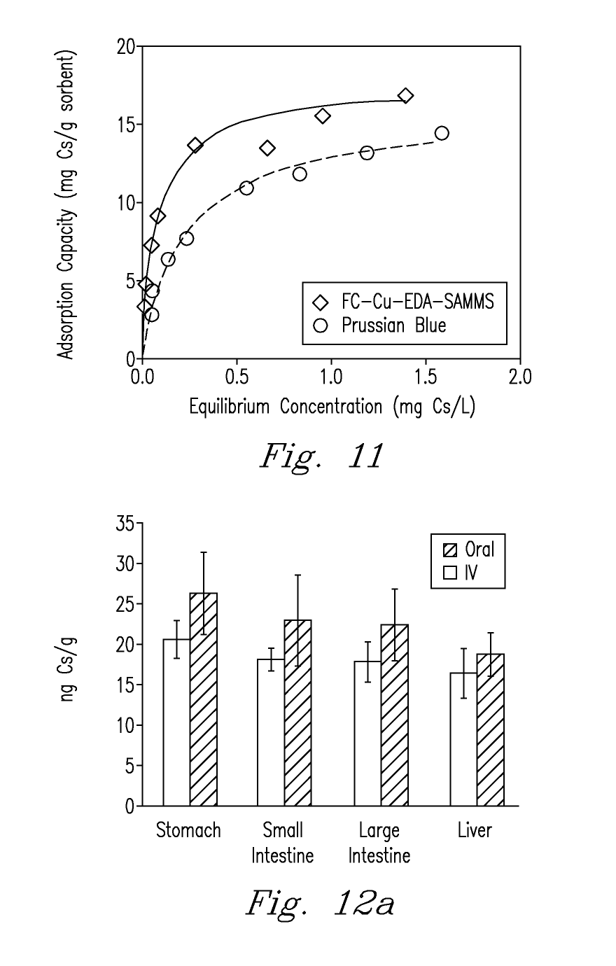

The adsorption isotherms on both sorbents are shown in FIGS. 10-11 for Cs in gastric and intestinal fluid stimulants, respectively. These adsorption isotherms were measured by increasing the loading of Cs in the simulants onto SAMMS or Prussian Blue while maintaining liquid-to-solid ratio of 10,000 mL/g. The plot between the equilibrium sorption capacities versus solution metal concentrations represents the adsorption isotherm curve. In gastric fluid simulant at low pH (1.1), SAMMS exhibited a very high maximum sorption capacity that exceeded Prussian Blue by an order of magnitude (21.7 vs. 2.6 mg Cs/g, respectively). In intestinal fluid stimulant (pH 8.6), SAMMS and Prussian Blue had a similar capacity (17.9 and 16.5 mg Cs/g, respectively).

### In Vivo

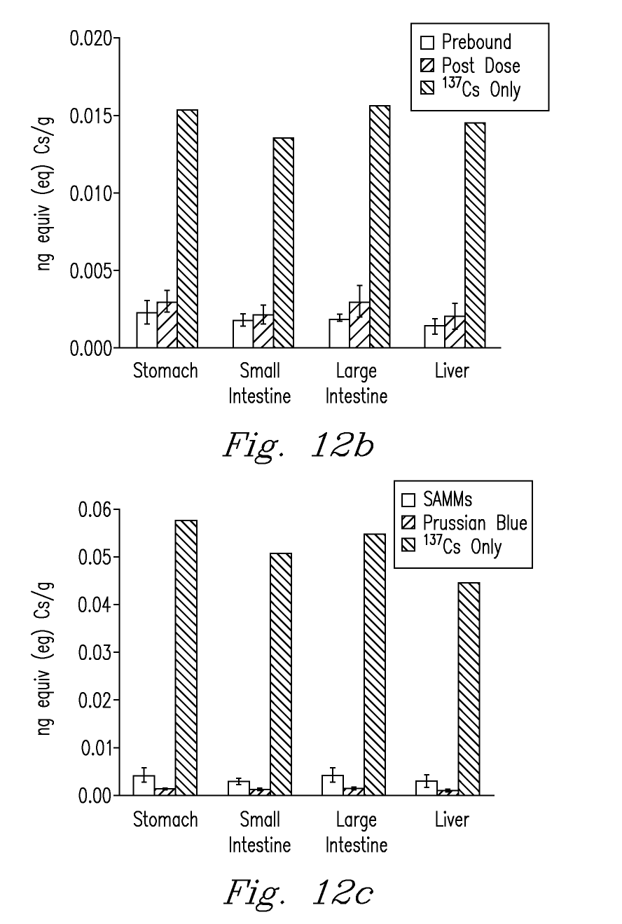

The pharmacokinetics of ¹³⁷Cs uptake, distribution and elimination were evaluated in rats following single dose exposures to ¹³⁷Cs (oral and iv), both in the presence or absence of decorporation agents (SAMMS & Prussian Blue). For all treatment groups (I→III), the time course of ¹³⁷Cs in selected tissues, excreta and calculated area-under-the-curve (AUC) are presented in FIGS. 12-15.

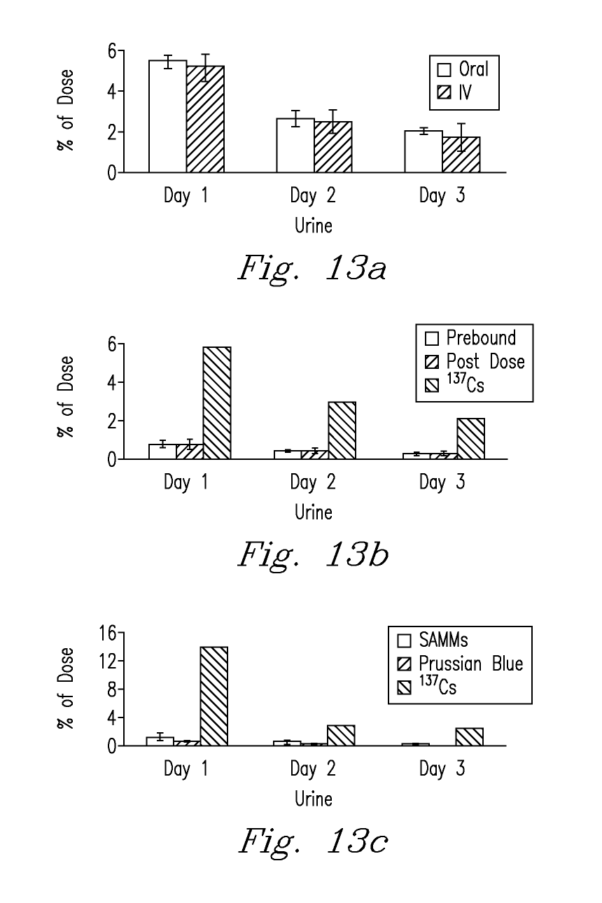

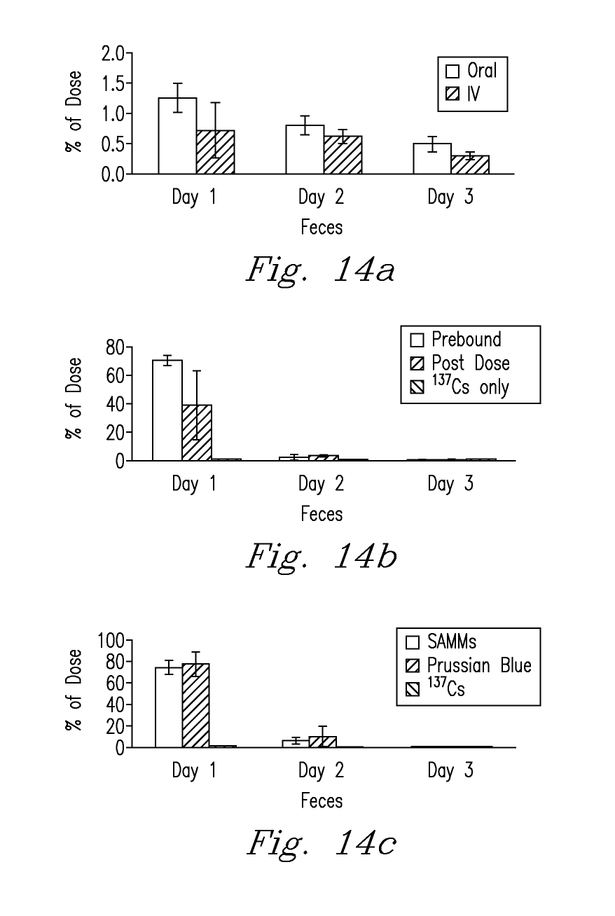

Group I. An evaluation of the pharmacokinetics following the equal molar ¹³⁷Cs doses via oral or iv administration strongly suggest that the kinetics are very comparable. For both dose routes, peak blood concentrations were observed at 0.5 hour and 24 hour post dosing which then gradually declined. The calculated AUC for the oral and iv groups are essentially the same (365-366 ng equiv/g/hr), which is consistent with the rapid and complete oral bioavailability of ¹³⁷Cs. A comparison of the ¹³⁷Cs concentration in gastrointestinal tract associated tissues/organs at 72 hours post-dosing are presented in FIG. 12a. The concentration of ¹³⁷Cs was very comparable in the stomach, small and large intestines, and liver, with oral administration resulting in a slightly lower tissue concentration (˜78-88%), relative to iv administration. The excretion time-course of ¹³⁷Cs in urine and feces are very comparable for the oral and iv doses and the results are presented in FIG. 13a and FIG. 14a. For both exposure routes, the urine is the predominant excretion pathway accounting for 18-20% of the dose; whereas, the feces only accounts for 2-3% (72 hour post-dosing). For both excretion pathways the first 24 hour collection interval (Day 1) accounted for the majority of ¹³⁷Cs that was excreted.

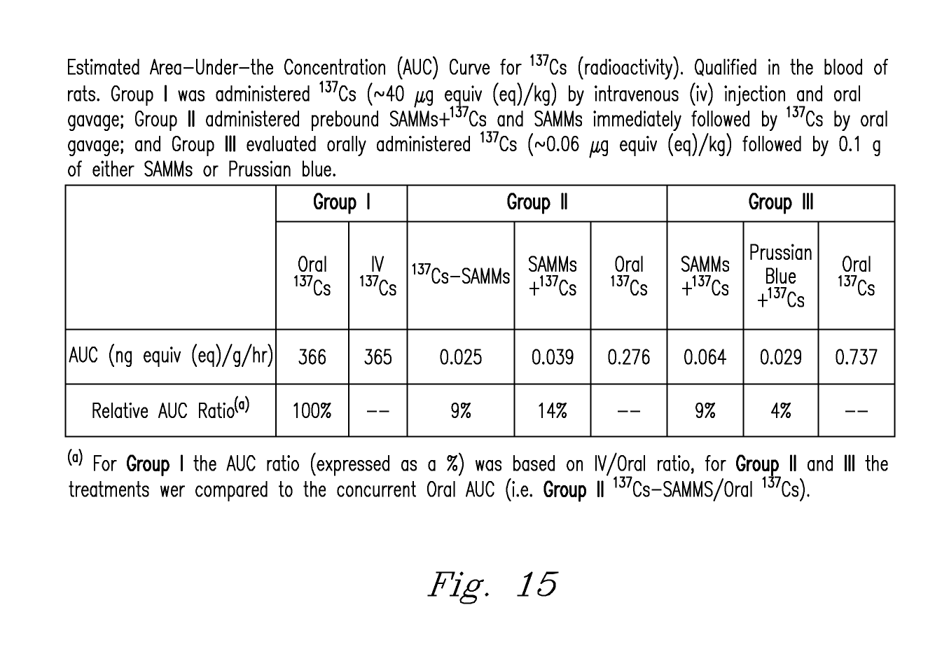

Group II. In these experiments equal molar doses of ¹³⁷Cs were administered to rats either pre-bound to SAMMS or the SAMMS was sequentially administered following the oral dose of ¹³⁷Cs. In addition, to facilitate comparison a single rat was administered ¹³⁷Cs only (no SAMMS). The time-course of ¹³⁷Cs in the blood and the calculated AUC are presented in FIG. 15. Although ¹³⁷Cs was detected in the blood following either SAMMS treatment, peak concentrations (24 hour post-dosing) range from 6- to 8-fold lower than what was observed for ¹³⁷Cs only. A comparison of the blood ¹³⁷Cs AUC suggests that 9% and 14% of the ¹³⁷Cs from the pre-bound and sequential SAMMS were absorbed, respectively. A comparison of the ¹³⁷Cs concentration in gastrointestinal tract associated tissues/organs at 72 hours post-dosing are presented in FIG. 12b. Consistent with the observed blood time-course results, the tissue concentration of ¹³⁷Cs were-10-fold lower for rats administered the pre-bound and sequential SAMMS, relative to the ¹³⁷Cs only. Following the SAMMS administrations (prebound & sequential), less than 1.5% of the administered dose of ¹³⁷Cs was accounted for in the urine of rats (through 72 hour post-dosing); whereas, for the ¹³⁷Cs only treatment, the urine accounted from >11% of the administered dose. In contrast, the pre-bound and sequential SAMMS treatments resulting in substantially more fecal excretion of ¹³⁷Cs, particularly in the first 24 hour where pre-bound and sequential administration accounted for 70 and 39% of the dose, respectively. In comparison less than 0.5% of the ¹³⁷Cs only dose was accounted for in the feces over the same collection interval. These results suggest that SAMMS binds rapidly with available ¹³⁷Cs in the gut and once ¹³⁷Cs is bound, it is stable and readily excreted in the feces.

Group III. In these experiments rats were orally administered equal molar doses of ¹³⁷Cs, then sequentially administered an oral dose (0.1 g) of either SAMMS or Prussian Blue and the pharmacokinetics of ¹³⁷Cs was evaluated. Again, to facilitate comparisons a single rat was administered ¹³⁷Cs only (no SAMMS or Prussian Blue). The time-course of ¹³⁷Cs in the blood and the calculated AUC are presented in TABLE 2. Both decorporation agents substantially decreased the ¹³⁷Cs blood concentration (10- to 100-fold) relative to ¹³⁷Cs only. Based on the blood time-course results and the calculated AUC, only 4% of the ¹³⁷Cs dose was absorbed following the Prussian Blue treatment, while SAMMS resulted in 9% absorption. The tissue concentrations of ¹³⁷Cs at 72 hour post-dosing are presented in FIG. 12c, and the tissue levels ranged from 20- to 60-fold less than what is observed following the ¹³⁷Cs only dose. In the absence of any decorporation agents the total amount of ¹³⁷Cs that was cumulatively excreted in the urine over 72 hours post-dosing was ˜20%; however, when either SAMMS or Prussian Blue were administered the total amount of radioactivity that was excreted in the urine was <2% (FIG. 13c). Consistent with the lack of urinary excretion of ¹³⁷Cs, an increase in the amount of ¹³⁷Cs eliminated via the feces was observed following SAMMS or Prussian Blue decorporation (FIG. 14c). Specifically an average of 80-90% of the ¹³⁷Cs was eliminated via the feces with the majority (74-78%) eliminated within the first 24 hours post-dosing for both decorporation agents. These results indicate that SAMMS can effectively decorporate ¹³⁷Cs when sequentially administered orally. At this dosage of ¹³⁷Cs, the in vivo efficacy of a single dose of FC-SAMMS (under current evaluation conditions) is comparable to Prussian Blue—the current “gold standard”.

However, even current Prussian Blue technology is not without faults. Of significant concern is the potential effect of low pH within the stomach. In this regard, it has been demonstrated that low pH can have a negative effect on the Prussian Blue binding of ¹³⁷Cs; however, the binding capacity of Prussian Blue rapidly recovers with increasing pH and maximum binding capacity is achieved within 4 hour at pH 5 (Faustino et al., J. Pharm. Biomed. Anal. 47: 114-25; 2008). The findings in the current study suggest that binding capacity of Prussian Blue is substantially decreased at low versus high pH (2.6 mg Cs/g vs. 16.5 mg Cs/g, respectively). In contrast, the maximum capacity of the SAMMS (22 mg Cs/g vs. 18 mg Cs/g) is not substantially impacted by pH. In the case of Prussian Blue, it has been suggested that Cs binding is reduced at low pH due to the greater availability of hydronium (H₃O⁺) ions, which compete with Cs⁺ ions for binding in the Prussian Blue lattice. In contrast, pH has little impact on the maximum binding capacity of SAMMS, suggesting that the FC—Cu-SAMMS is not protonated to the degree that Prussian Blue is at the low pH that is encountered in the stomach.

### CONCLUSIONS

The current study has established: 1) that SAMMS can rapidly decorporate ¹³⁷Cs following oral administration and 2) that the SAMMS-¹³⁷Cs complex is very stable in the GI tract. These findings are the first to establish the binding stability of SAMMS in vivo in the GI tract (i.e., at low to high pH).

While various preferred embodiments of the invention are shown and described, it is to be distinctly understood that this invention is not limited thereto but may be variously embodied to practice within the scope of the following claims. From the foregoing description, it will be apparent that various changes may be made without departing from the spirit and scope of the invention as defined by the following claims.

We claim:

1. An insoluble therapeutic sorbent for removing materials from a biological system, said therapeutic sorbent comprising:
a material having a rigid, porous backbone coupled to at least one ligand configured to attach to a particular preselected target.

2. The insoluble therapeutic sorbent of claim 1, wherein said material comprises ligands specific to at least two preselected targets.

3. The insoluble therapeutic sorbent of claim 1, wherein said material is embodied in an oral delivery device.

4. The insoluble therapeutic sorbent of claim 1, wherein said material is embodied in a dermal delivery device.

5. The insoluble therapeutic sorbent of claim 1, wherein said material is embodied in a blood filtering system.

6. The insoluble therapeutic sorbent of claim 1, wherein said material is a functionalized mesoporous silica.

7. The insoluble therapeutic sorbent of claim 1, wherein said material is a chemically modified activated carbon.

8. The insoluble therapeutic sorbent of claim 1, wherein said material is a functionalized mesoporous carbon.

9. The insoluble therapeutic sorbent of claim 1, wherein said target is selected from the group consisting of: Cd, Pb, Hg, Tl, As, Gd, phosphate, U, Pu, Am, Cs, and Co.

10. The insoluble therapeutic sorbent of claim 1, wherein said target is a biological waste.

11. An insoluble therapeutic chelating agent for removing heavy metals and radionuclide materials from a biological system, said therapeutic chelating agent comprising:
a material having a rigid porous back bone coupled to at least one ligand configured to attached to a particular preselected target.

12. The insoluble therapeutic chelating agent of claim 11, wherein said material comprises ligands specific to at least two preselected targets.

13. The insoluble therapeutic chelating agent of claim 11, wherein said material is embodied in an oral delivery device.

14. The insoluble therapeutic chelating agent of claim 11, wherein said material comprises a functionalized mesoporous silica.

15. The insoluble therapeutic chelating agent of claim 11, wherein said material is a chemically modified activated carbon.

16. The insoluble therapeutic chelating agent of claim 11, wherein said material comprises a functionalized mesoporous carbon.

17. The insoluble therapeutic chelating agent of claim 11, wherein said target is selected from the group consisting of: Cd, Pb, Hg, Tl, As, Gd, phosphate, U, Pu, Am, Cs, and Co.

18. A method for removing target materials from a biological system, characterized by:
administering an oral dosage of an insoluble therapeutic chelating agent, said insoluble therapeutic chelating agent comprises a material having a rigid, porous backbone coupled to at least one ligand that is configured to attach to a particular preselected target.

19. The method of claim 18, wherein said preselected target is captured by said therapeutic chelating agent in the GI tract and excreted fecally.
